(19) United States
(12) Patent Application Publication
(10) Pub. No.: US 2012/0053527 A1
(43) Pub. Date: Mar. 1, 2012

(54) MEDICATION INJECTION SUPERVISOR DEVICE

(76) Inventors: William Cirillo, Dublin (IE); John Hughes, Dublin (IE)

(21) Appl. No.: 13/291,073

(22) Filed: Nov. 7, 2011

Related U.S. Application Data
(63) Continuation-in-part of application No. 12/724,411, filed on Mar. 15, 2010.
(60) Provisional application No. 61/175,810, filed on May 6, 2009.

(51) Int. Cl. A61M 5/31

(52) U.S. Cl. 604/189

(57) ABSTRACT

Provided are medication injection supervisor devices for use with prefabricated injection pens. A medication injection supervisor device may include a universal header having one or more attachment features for attaching the header to a prefabricated injection pen. The attachment may be removable or permanent. Some examples of removable attachments include a sleeve for positioning around the pen, a slot for receiving a ridge of the pen, a cavity for receiving a clip or a specially configured pin of the pen, and two rings into which the pen may be received. Some examples of permanent attachments include integrated bodies of the pen and universal header and glue. The medication injection supervisor device may also include an electronics assembly housed by the universal header, a sensor for automatic detection of an injection, a display for viewing injection data, and one or more buttons for manipulating the injection data, such as resetting the time.

### RELATED APPLICATIONS

This application is a continuation in part of U.S. patent application Ser. No. 12/724,411, filed on Mar. 15, 2010, which claims the benefit of the filing date of Provisional Application 61/175,810 filed on May 6, 2009. Both of these applications are incorporated herein by reference in their entirety for all purposes.

### FIELD

The present invention relates generally to devices for management of chronic medical conditions requiring periodic administration of medications by self-injection. More specifically, the present invention relates to an assembly adaptable to a variety of medication injection delivery devices.

### BACKGROUND

To maintain optimum conditions, a patient suffering from a chronic medical condition is required to conform to a prescribed administration schedule of a medication, adhere to a prescribed dosage, avoid extra administrations, avoid missed administrations, and adhere to various recommended health and safety best practices.

The long-term health of the patient with a chronic medical condition depends on the day-to-day management of the condition. Mismanagement of the condition can result in significant morbidity and mortality and carry an increased risk of developing complications. A focused approach to the management of a chronic medical condition is essential for the patient in order to reduce the occurrence of these complications.

For example, diabetes occurs when the body does not produce enough insulin resulting for many diabetics in a requirement of a periodic insulin injection to control glucose levels in the body. One of the dangers associated with controlling glucose levels with insulin is insulin overdose. Symptoms of an insulin overdose reflect low blood sugar levels (hypoglycemia) and can include headache, irregular heartbeat, increased heart rate or pulse, sweating, tremor, nausea, increased hunger, and anxiety.

An insulin dependent diabetic needs to keep accurate track of the type and amount of insulin he is injecting. Individual insulin products are numerous, but currently insulin may be divided into four major types: 1) Short-acting insulin, which is soluble and acts quickly (within 30-60 minutes) and lasts between 6 and 8 hours. Some subtypes of this soluble insulin may act faster and last for a shorter time. 2) Intermediate-acting insulin (isophane insulin), which acts slightly more slowly (within 1-2 hours) and lasts between 10 and 14 hours. 3) Long-acting insulin such as determir, glargine, protamine zinc, and zinc suspension, which acts comparatively slowly (1-2 hours) and lasts comparatively much longer, for up to 24 hours. 4) Various mixtures of the above-mentioned three major types of insulin. Different amounts of the short and intermediate-acting insulin can be mixed together depending on the requirements of an individual case. A user can suffer long-term health consequences if too little insulin is taken. If, on the other hand, too much insulin is taken, the user can suffer immediate hypoglycemia leading to coma and hospitalization.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

Provided are medication injection supervisor devices for use with a prefabricated injection pen. A medication injection supervisor device may include a universal header having one or more attachment features for attaching the header to a prefabricated injection pen. The attachment may be removable or a permanent feature of the prefabricated injection pen. Some examples of removable attachments include a sleeve for positioning around the pen, a slot for receiving a ridge of the pen, a cavity for receiving a clip or a specially configured pin of the pen, and two rings for receiving the pen. Some examples of permanent attachments include integrated bodies of the pen that contain permanent features to which a header can be attached or universal header and glue. The medication injection supervisor device may also include an electronics assembly housed in the universal header, a sensor for automatic detection of an injection, a display for viewing injection data, and one or more buttons for manipulating the injection data (such as resetting the time).

In certain embodiments, a medication injection supervisor device includes a universal header including one or more attachment features for attaching the universal header to a prefabricated injection pen. The medication injection supervisor device also includes an electronics assembly housed by the universal header, a sensor to automatically detect an injection such that the sensor is communicatively coupled to the electronics assembly, a display to display injection data, and one or more buttons to allow the manipulation and display of the injection data (such as resetting time).

In certain embodiments, the one or more attachment features are configured to provide a removable attachment of the universal header to the prefabricated injection pen. For example, the one or more attachment features may include a sleeve having an inner surface. The inner surface is configured to come in contact with the prefabricated injection pen when the universal header is attached to the prefabricated injection pen. The inner surface may be or include a friction-generating surface. In certain embodiments, the inner surface follows the shape of the prefabricated injection pen. The sleeve may be removably attached to the universal header. The sleeve may include two flexible portions forming a partial circumference having an opening and wherein flexing the flexible portions changes the size of the opening.

In certain embodiments, one or more attachment features include a slot for receiving a ridge of the prefabricated injection pen. An opening of the slot may be narrower than a bottom of the slot to prevent the ridge from sliding out of the slot in a direction substantially perpendicular to the bottom. The slot may include one or more retaining features to prevent the ridge from sliding out of the slot in a direction substantially parallel to a bottom of the slot.

In certain embodiments, the universal header includes a cavity for receiving a clip of the prefabricated injection pen. The cavity is or is a part of the one or more attachment features. The clip of the prefabricated injection pen may be similar to clips of conventional writing pens and may be used, when not inserted into the cavity of the universal header, for supporting the prefabricated injection pen in pockets or to affix sheets of paper to the pen.

In certain embodiments, the universal header includes a cavity for receiving a pin of the prefabricated injection pen. A cavity-pin coupling is or is a part of the one or more attachment features. In the same or other embodiments, the universal header includes a pin for inserting into a cavity of the prefabricated injection pen.

In certain embodiments, the one or more attachment features include at least two rings affixed to the universal header and configured for receiving and tightly fitting the prefabricated injection pen into the two rings. The two rings may be spring rings configured to change their diameters upon inserting forces onto the two rings when inserting the prefabricated injection pen into the two rings.

In certain embodiments, the universal header includes a surface for contacting the prefabricated injection pen such that the surface conforms to a profile of the prefabricated injection pen. The one or more attachment features include a double-sided tape positioned on the surface of the universal header.

Also provided is a medication injection supervisor assembly that includes a prefabricated injection pen, a universal header permanently attached to the prefabricated injection pen, an electronics assembly housed by the universal header, and a sensor to automatically detect an injection such that the sensor is communicatively coupled to the electronics assembly, a display to display injection data, and one or more buttons to allow manipulation and display of the injection data (such as resetting the time). The universal header may be permanently attached to the prefabricated injection pen using an adhesive. In other embodiments, the universal header forms a joined body with the prefabricated injection pen.

These and other embodiments are described further below with reference to the figures.

### BRIEF DESCRIPTION OF DRAWINGS

Example embodiments are illustrated by way of example and not limitation in the figures of the accompanying drawings, in which like references indicate similar elements and in which:

FIG. 1 is a perspective view of a medication injection supervisor device, in accordance with an example embodiment.

FIG. 2 shows the measurement history of a medication injection supervisor device, in accordance with an example embodiment.

FIG. 3 shows various messages that may appear on the display screen of a medication injection supervisor device, in accordance with an example embodiment.

FIG. 4 is a perspective view of plunger break or locking pin device of a medication injection supervisor device, in accordance with an example embodiment.

FIG. 5 is a perspective view of a medication injection supervisor device mounted on an injection pen, in accordance with an example embodiment.

FIG. 6 is an inverted view of a universal header, in accordance with an example embodiment.

FIG. 7 is a sleeve specific to an injection pen model, in accordance with an example embodiment.

FIG. 8 is a universal header being mounted on a sleeve, in accordance with an example embodiment.

FIG. 9 is a universal header mounted on a sleeve, in accordance with an example embodiment.

FIG. 10 is an empty universal header and sleeve assembly mounted on an injection pen with locking indents visible, in accordance with an example embodiment.

FIG. 11 is an exploded view of a medication injection supervisor device, in accordance with an example embodiment.

FIG. 12 shows a sleeve being mounted on a Sanofi Aventis Lantus SoloStar injection pen, in accordance with an example embodiment.

FIG. 13 shows a universal header being installed on a sleeve mounted on a Sanofi Aventis Lantus SoloStar injection pen, in accordance with an example embodiment.

FIG. 14 shows a sleeve being mounted on a Novo Nordisk FlexPen injection pen, in accordance with an example embodiment.

FIG. 15 shows a universal header being installed on a sleeve mounted on a Novo Nordisk FlexPen injection pen, in accordance with an example embodiment.

FIG. 16 is a flow diagram showing a method for supervising injections using the medication injection supervising device, in accordance with an example embodiment;

FIG. 17 shows a readable plunger subsystem, in accordance with an example embodiment.

FIG. 18 shows a micro optics and imaging system, in accordance with an example embodiment.

FIG. 19 is a cross-sectional schematic view of a medication injection supervisor assembly, in accordance with an example embodiment.

FIG. 20 is a cross-sectional schematic view of a medication injection supervisor assembly, in accordance with a different example embodiment.

FIG. 21 is a cross-sectional schematic view of a medication injection supervisor assembly, in accordance with a different example embodiment.

FIG. 22 is a cross-sectional schematic view of a medication injection supervisor assembly, in accordance with a different example embodiment.

FIG. 23A is a cross-sectional schematic view of a medication injection supervisor assembly, in accordance with a different example embodiment.

FIG. 23B is a side schematic view of the medication injection supervisor assembly shown in FIG. 23A, in accordance with a different example embodiment.

### DETAILED DESCRIPTION

The following detailed description includes references to the accompanying drawings, which form a part of the detailed description. The drawings show illustrations in accordance with example embodiments. These example embodiments, which are also referred to herein as “examples,” are described in enough detail to enable those skilled in the art to practice the present subject matter. The embodiments can be combined, and other embodiments can be formed, by introducing structural, logical or electrical changes without departing from the scope of what is claimed. The following detailed description is, therefore, not to be taken in a limiting sense, and the scope is defined by the appended claims and their equivalents.

In this document, the terms “a” or “an” are used, as is common in patent documents, to include one or more than one. In this document, the term “or” is used to refer to a nonexclusive “or,” such that “A or B” includes “A but not B,” “B but not A,” and “A and B,” unless otherwise indicated. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In the example embodiments described herein, the medication injection supervisor device is an attachment to a standard injection pen, which is sufficiently small to be carried by patients in a pocket or a purse. The medication injection supervisor device can allow patients to keep accurate injection records. In addition to providing a method for keeping one's medication injection records, the medication injection supervisor device can provide reminders and other warnings about events that may affect a patient's health.

In some example embodiments, the medication injection supervisor device can record events and generate reports to spot trends and patterns so that adjustments can be made to medication dosage, exercise, or eating. This kind of record keeping can provide someone with a chronic medical condition with a long-term view of his disease that will in turn help him control the disease and lead a healthier life without complications therefrom.

The medication injection supervisor device, according to an example embodiment, can be mounted on a variety of prefabricated injection pens to record the time since the last injection. The device can automatically trigger whenever the user injects the medication and thus can provide a reliable means to note the time elapsed since the last injection. Prefabricated injection pens can be of various shapes, depending on the manufacturer. To accommodate these various shapes, a two-part attachment method can be used. The first part can be a sleeve designed to lock solidly and robustly onto the particular injection pen. The second part can be a universal header attached to the sleeve unit and housing the electronics.

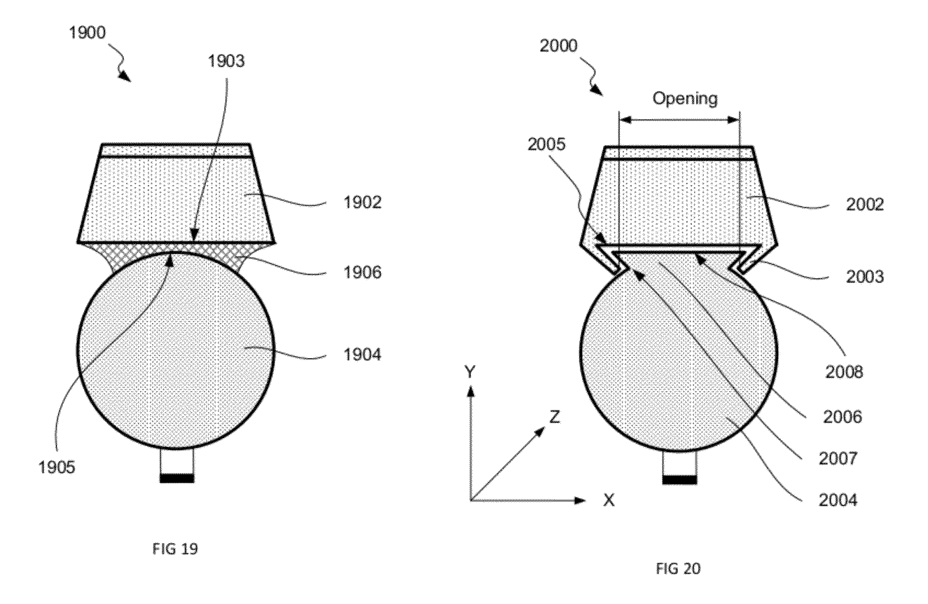

A medication injection supervisor device may include a universal header having one or more attachment features for attaching the header to a prefabricated injection pen. The attachment may be removable or permanent. Various examples of these attachment features are described below with reference to FIGS. 19-23B.

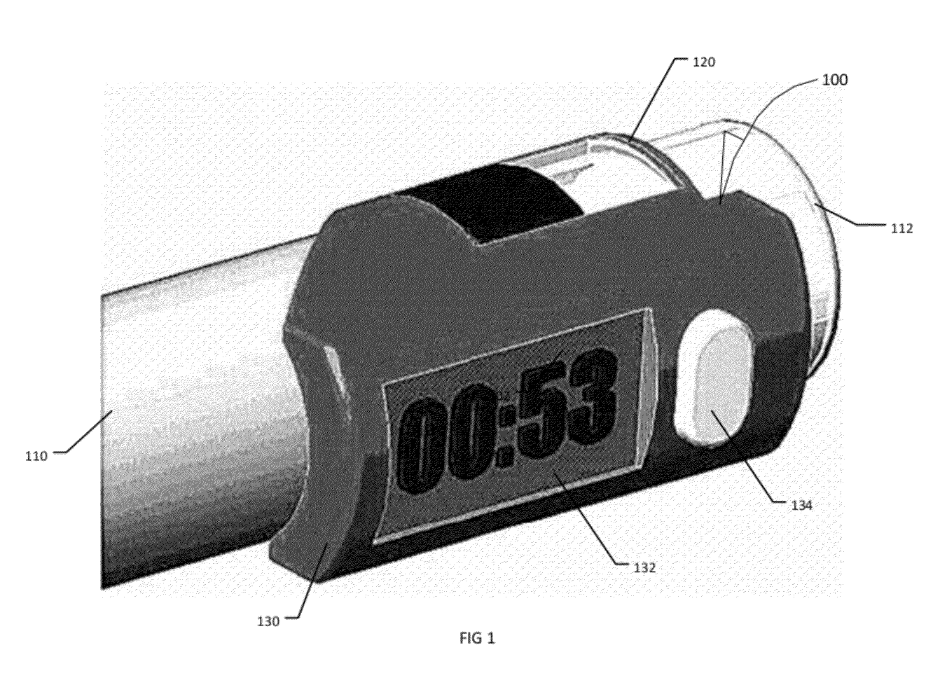

FIG. 1 is a perspective view of a medication injection supervisor device 100, in accordance with an example embodiment. As shown in FIG. 1, the medication injection supervisor device 100 includes a sleeve 120 and a universal header 130. An injection pen 110 is prefabricated and is not a part of the medication injection supervisor device 100. The injection pen 110 can include a plunger 112, which can be extended to dial a medication dosage. The universal header 130 can include a display 132 to display various data related to medication injection supervision. For example, as shown in FIG. 1, the display 132 can display the time since last injection. The universal header 130 can also include one or more buttons 134 to allow the user to manipulate and display the data related to medication injection supervision.

In some example embodiments, the one or more buttons 134 can be used to turn on the display 132. In some example embodiments, it is not necessary to utilize the one or more buttons 134 to turn off the display 132, which turns off automatically after a certain period of time. In some example embodiments, the display 132 does not display the time of day, as the time displayed is elapsed time only. Furthermore, there is no requirement for start and end times of injections to be displayed.

As shown in FIG. 1, the sleeve 120 is adapted to the injection pen 110, and the universal header 130 is locked onto the sleeve 120 at locking indents (not shown). This approach allows the universal header 130 to be mounted on a variety of existing injection pens. Thus, the medication injection supervisor device 100 facilitates a two-part attachment method, wherein the sleeve 120 is customized to fit a specific model of an injection pen. This allows the universal header 130 that houses the electronics to be used on a variety of injection pens. Additionally, this allows for easy attachment and removal of the medication injection supervisor device 100, and for its precise positioning for various injection pen types without complex steps. In some example embodiments, the medication injection supervisor device 100 is fully automatic and requires no user input so that there is no need to confirm any action and/or to record an event.

The medication injection supervisor device 100 can be characterized as a clip-on injection recorder that attaches to the injection pen 110 to assist in maintaining optimum patient control. The medication injection supervisor device 100 is a clip-on designed to fit an injection pen of a specific brand. The header 130, shown in FIG. 1, can be robust enough for regular use and simple enough for very young children and the elderly to fit and remove. The design of the medication injection supervisor device 100 is slim and lightweight so as not to increase the total bulkiness of the injection pen 110 significantly.

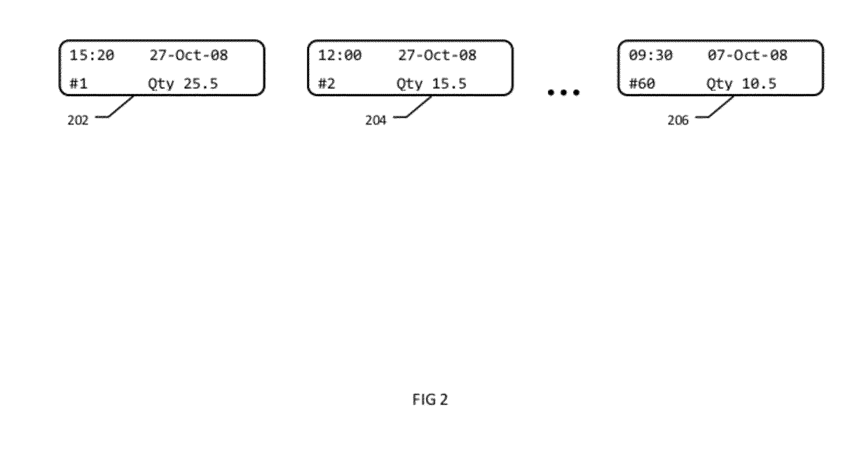

FIG. 2 shows the measurement history of the medication injection supervisor device 100, in accordance with an example embodiment. As shown in FIG. 2, the medication injection supervisor device 100 can record the dosage and time of each injection (202, 204, and 206), along with any warnings, over a predetermined period of time. For example, for regular usage, there can be up to four readings a day for thirty days. A user can have the ability to set up to four injection times and tolerances, with these being the “scheduled” times the user is due to inject.

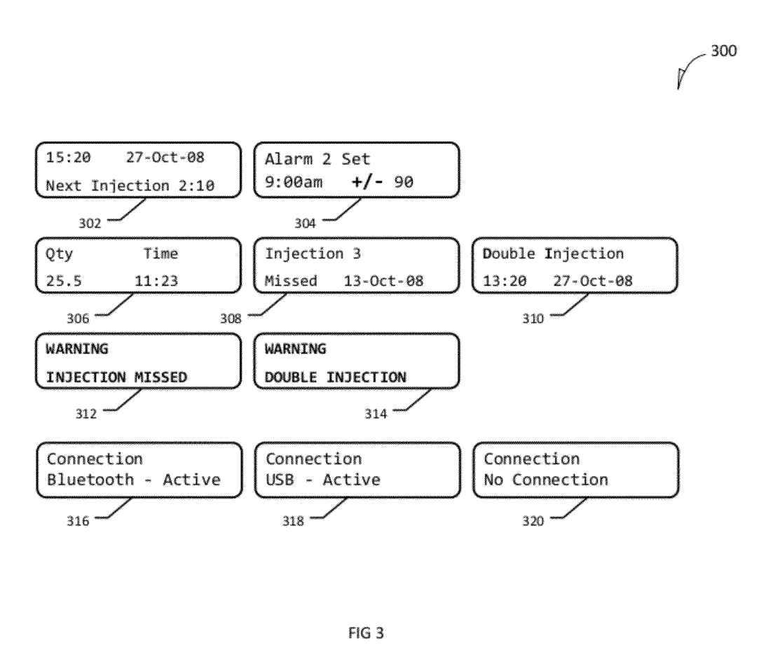

FIG. 3 shows various messages that may appear on the display 132 of the medication injection supervisor device 100, in accordance with an example embodiment. The display 132, as shown in FIG. 3, allows the user to step through the memory of recorded values directly from the medication injection supervisor device 100 as well as view any warnings or messages. The medication injection supervisor device 100 has the ability to issue an alarm in both an auditory and a visual manner after the occurrence of one or more of the following: a missed injection, an injection dosage outside of the expected range, and a “double” injection. The medication injection supervisor device 100 can also check for and detect certain situations where a warning is to be provided. For example, the medication injection supervisor device 100 can warn when the cartridge of the medication pen 110 is low or the pen temperature has exceeded a recommended safe range for the medication. The alarm may also be used to provide an alert, a reminder, or other scheduled event such as an “injection due” reminder or a reminder to change the needle after a given number of injections or uses.

The alarm features of the medication injection supervisor device 100 can address all of these points in a way that extends the basic ideal of a simple alarm. In this regard, the device can learn from a past usage pattern and can start to predict optimum injection times and suggest dosage. Thus, the device can monitor for dangerous usage (e.g., injecting a larger than recommended dosage or multiple dosages in a too-short period of time).

The device can also be comprised of a built-in SMS capability to automatically send injection usage to a “caregiver,” to allow remote confirmation and monitoring for parents of young children as well as those looking after the elderly; to send general summary information concerning daily or weekly usage to nominated numbers; and to send real-time emergency SMS text messages to report “double dosage” to inform of a potential risk.

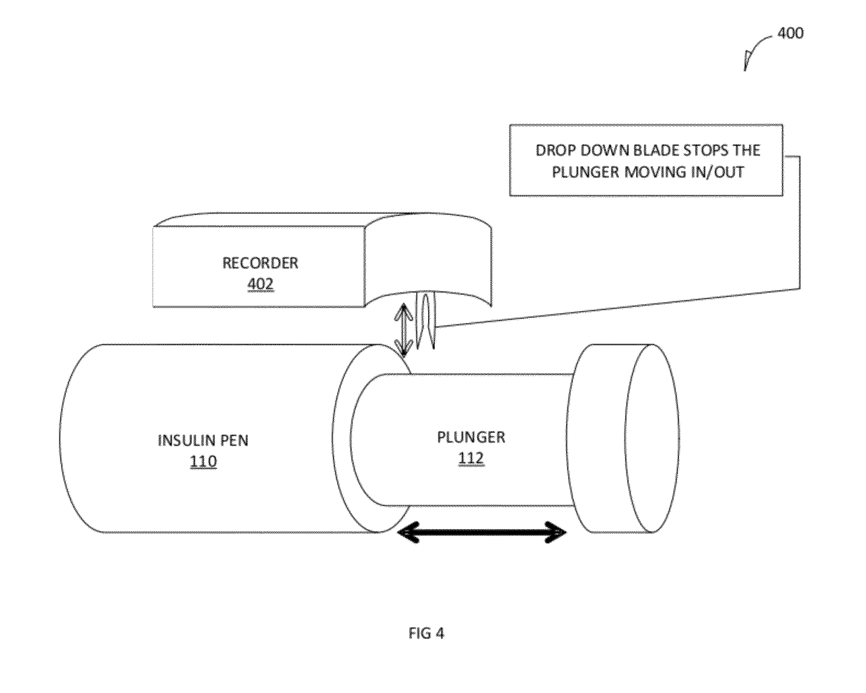

FIG. 4 is a perspective view of a plunger break or locking pin device of the medication injection supervisor device 100, in accordance with an example embodiment. In some example embodiments, when the medication injection supervisor device 100 detects a “dangerous situation” (e.g., too high of a dose), not only can it generate an auditory and visual alarm, but it can also activate a locking pin, as shown in FIG. 4, that will prevent the user from pressing the plunger 112. This pin can be spring loaded and designed such that the user dials a lower dose before the pin is released and the user is allowed to proceed with the injection.

The medication injection supervisor device 100 may also include an interface to download data to and configure the device from a personal computer (PC). As the user builds up usage information over a number of weeks, this data is analyzed via separate software on the PC to produce trend information. This can be used to allow the user to “optimize” his medication usage, and is “fed back” into the pen to suggest the “expected” dosage and warn if the user is deviating significantly from it. This information can also be used by a medical practitioner to analyze any change in the user's condition.

The SMS functionality can allow a third party to be informed of any potentially dangerous situations concerning medication levels and, generally, to allow monitoring of whether the medication user is keeping to his recommended routine. This can be important when looking after young children and the elderly. The SMS feature can be extended further to allow text messages to be sent to the injection pen 110 to be displayed to the user on the display 132 should the usage data that has been downloaded indicate that a change in routine is required.

In some example embodiments, the user can step through the memory of recorded values, warnings, and messages directly from the device using the one or more buttons 134. The device can have the ability to alarm in both an auditory and visual manner after the occurrence of missed injections, injection dosages being outside the expected range, and “double” injections. The device can also check for and detect certain situations where a warning is to be provided. For example, the device can warn when the cartridge is low or the pen temperature has exceeded its recommended safe range for medication.

In some example embodiments, the alarm may also be used to provide an alert for reminders or other scheduled events, such as an “injection due” reminder and a reminder to change the injection pen's needle after a given number of injections or uses. Furthermore, the device can learn from a past usage pattern and can start predicting optimum injection times and suggest dosage amounts. Additionally, the device can monitor for dangerous usage (e.g., injecting a larger than recommended dosage or multiple dosages in too-short a period of time).

In some example embodiments, the device can facilitate remote monitoring of the patient's condition by sending information wirelessly. For example, the device can send information concerning injection usage to allow remote confirmation and monitoring to parents of young children and those who care for the elderly, send general summary information on daily or weekly usage to nominated numbers, and send instant emergency messages to report “double dosage” to inform of a potential risk.

In some example embodiments, the device can include an interface for downloading data to configure the device. As the user builds up usage information over a period of time, this data can be analyzed via separate software on a computing device to produce trend information. This information can be used to enable the user to optimize his medication usage, and can be fed back into the pen to suggest a dosage and warn if the user's usage is deviating significantly.

FIG. 5 is a perspective view of the medication injection supervisor device 100 installed on the injection pen 110, in accordance with an example embodiment. As shown, the medication injection supervisor device 100 can include the sleeve 120 and the universal header 130. The universal header 130 can, in turn, include the display 132 and the one or more buttons 134. The universal header 130 can be mounted on the sleeve 120 by sliding the universal header 130 on rails of the sleeve 120 designed to accommodate the universal header 130. When the universal header 130 is so mounted, the sleeve 120 squeezes the injection pen 110 and holds the medication injection supervisor device 100 tightly in place.

Thereafter, the medication injection supervisor device 100, according to an example embodiment, can assist in the control of medication injections by recording, monitoring, recommending, reporting, and protecting a user. The medication injection supervisor device 100 can be small enough so one can easily carry it with him wherever he goes as an attachment to a standard injection pen. In addition to providing a quick and easy method of recording his medication injection history, a user can be provided with a reminder or a warning if those readings were fed into the universal header 130 so that adjustments can be made in medication, exercise, or eating. This approach can provide someone with a chronic condition with a long-term view of his disease that will in turn help him control the disease and lead a healthier life without complications related thereto.

The medication injection supervisor device 100, according to an example embodiment, may require no user input because there is no need to confirm any action or to record any event. Thus, the medication injection supervisor device 100 can be fully automated. The medication injection supervisor device 100 can include only one button, which is used to turn on the display 132. The medication injection supervisor device 100 can record the dosage and time of each injection along with any warnings over a time period. It can have the ability to set a plurality of injection times and tolerances, which are the scheduled times the user is due to inject.

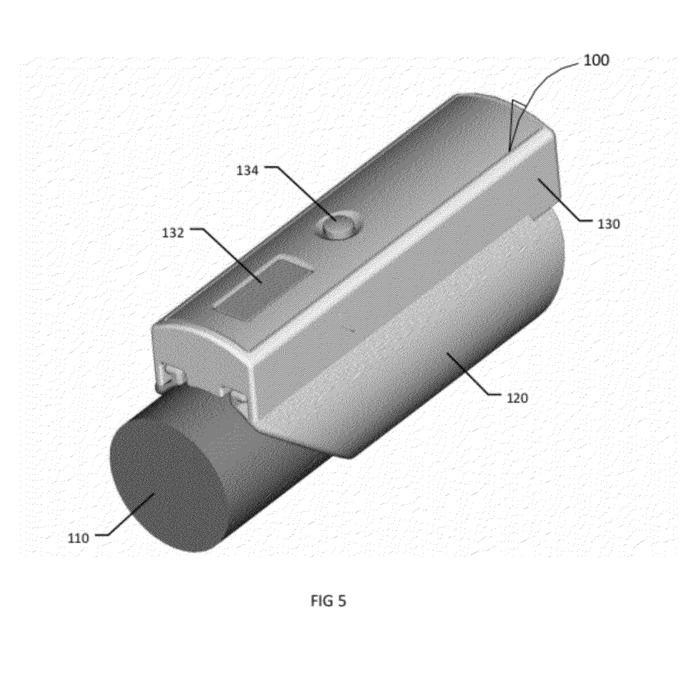

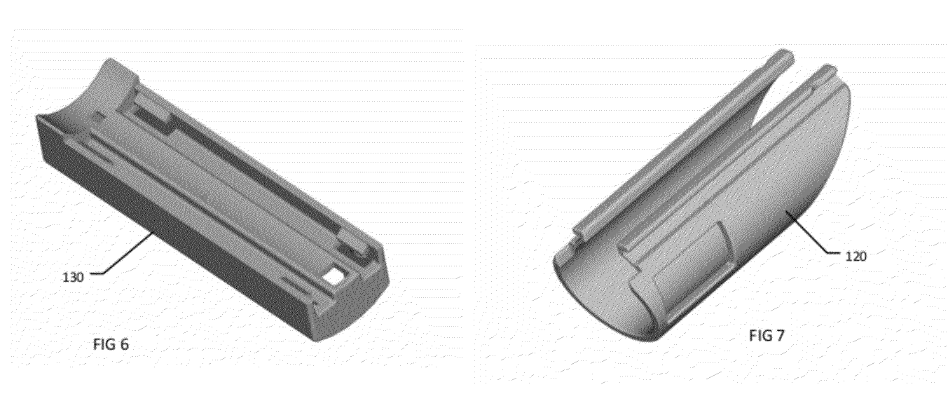

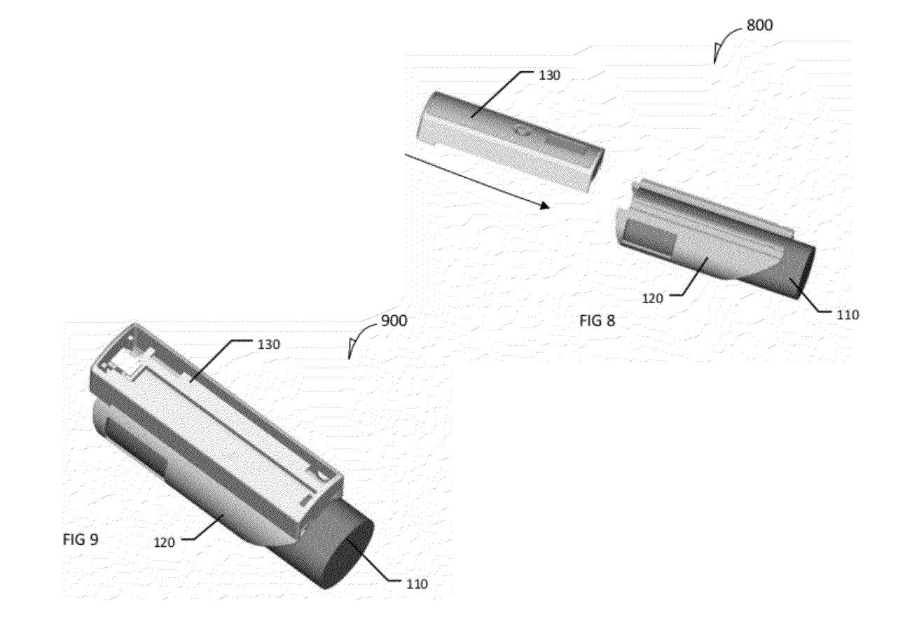

FIG. 6 is an inverted view of the universal header 130, in accordance with an example embodiment. FIG. 7 is the sleeve 120 specific to each injection pen model, in accordance with an example embodiment. FIG. 8 is the universal header 130 being mounted on a specific sleeve 120, in accordance with an example embodiment. FIG. 9 is the universal header 130 mounted on a specific sleeve assembly, in accordance with an example embodiment. The medication injection supervisor device 100 can be mounted on a variety of prefabricated injection pens to record the time since last injection. This information assists the user of the prefabricated injection pen in maintaining optimum diabetic control.

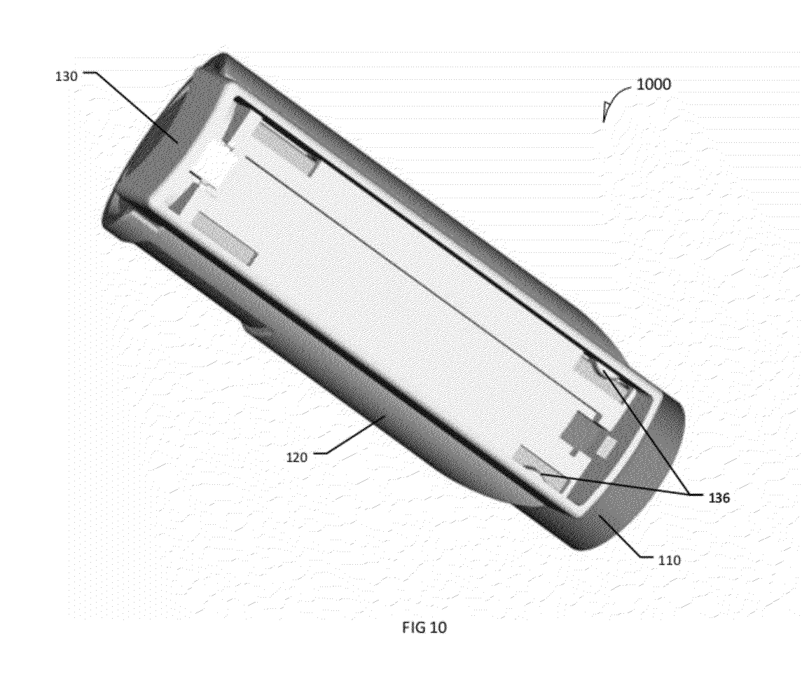

The medication injection supervisor device 100 automatically triggers whenever the user injects, and thus provides a simple and reliable means to note the elapsed time since the last injection. The prefabricated injection pens can be of various shapes, depending on their respective manufacturers. To accommodate these various shapes, a two-part attachment method is used. The first part is a sleeve designed to lock solidly and robustly onto the particular injection pen. The second part is a universal header unit attached to the sleeve that houses the electronics. FIG. 10 is an empty universal header and specific sleeve assembly mounted on a specific injection pen model with the locking indents 136 visible, in accordance with an example embodiment.

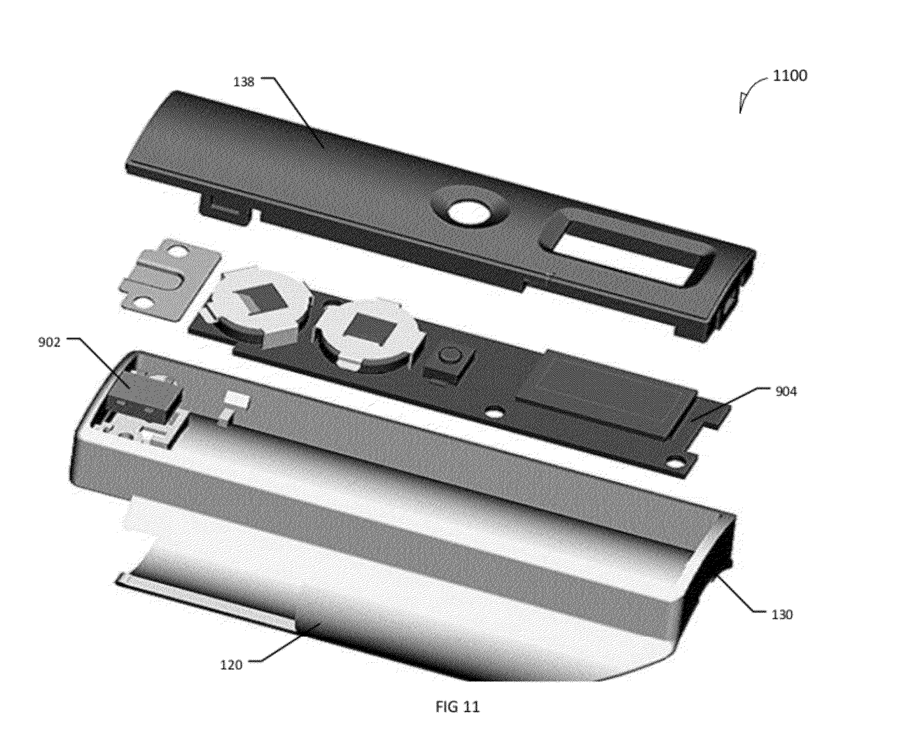

FIG. 11 is an exploded view of the medication injection supervisor device 100, in accordance with an example embodiment. As shown, the medication injection supervisor device 100 can include a cover plate 138, an electronic assembly 904, a sensor 902, a medication pen, the universal header 130, and the sleeve 120. In some example embodiments, when the medication injection supervisor device 100 is mounted on an injection pen 110, it positions the sensor 902 over the edge of the plunger (not shown) of the injection pen 110. In most injection pens, users extend the plunger to dial a dosage. When the plunger is extended, the sensor 902 (e.g., a micro switch) is toggled to the open state. When the sensor 902 is toggled to the open state, the timer is reset to zero to indicate that the injection has started.

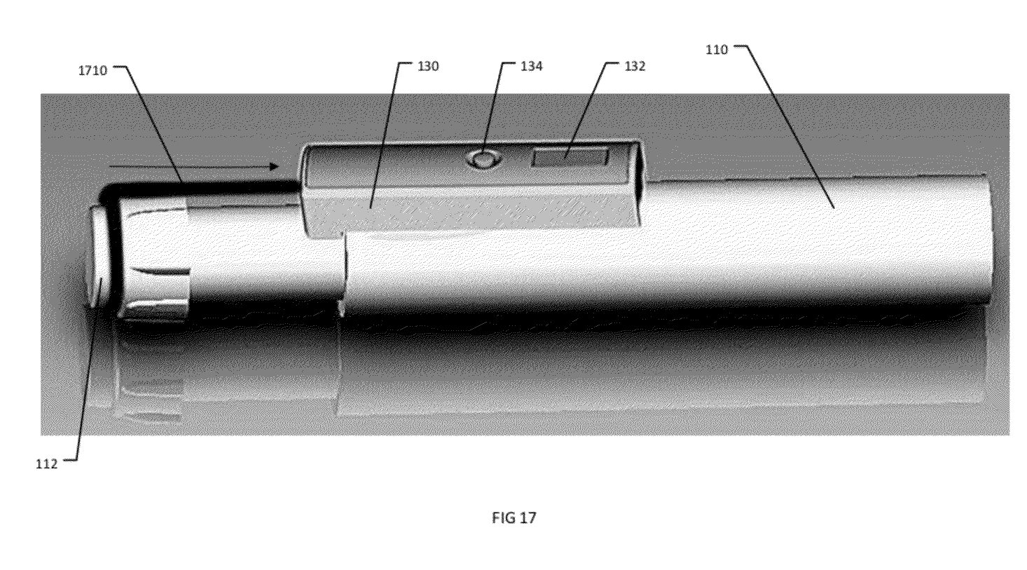

When the plunger is pressed to complete the injection, the sensor 902 is closed and the timer starts counting. In some example embodiments, a sound-generating unit (not shown) and/or a vibrational unit (not shown) can be installed to alert the user. It will be understood that some injection pens may not use a plunger to facilitate injections. Therefore, other techniques can be used to determine the occurrence of an injection and/or the dosage. For example, a micro optics and imaging system, as described below with reference to FIG. 17, can be used to determine the occurrence of an injection and/or the dosage.

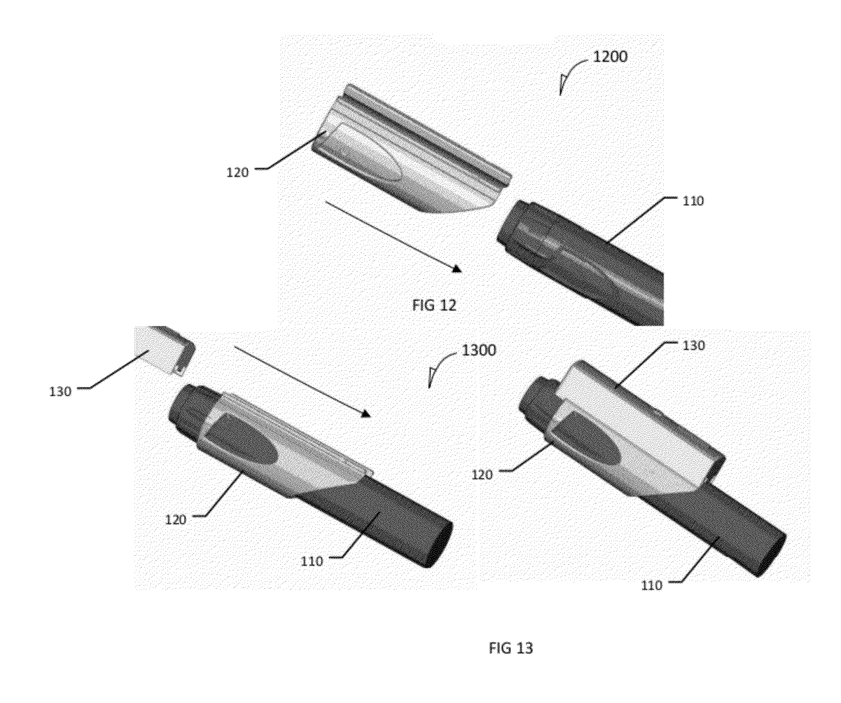

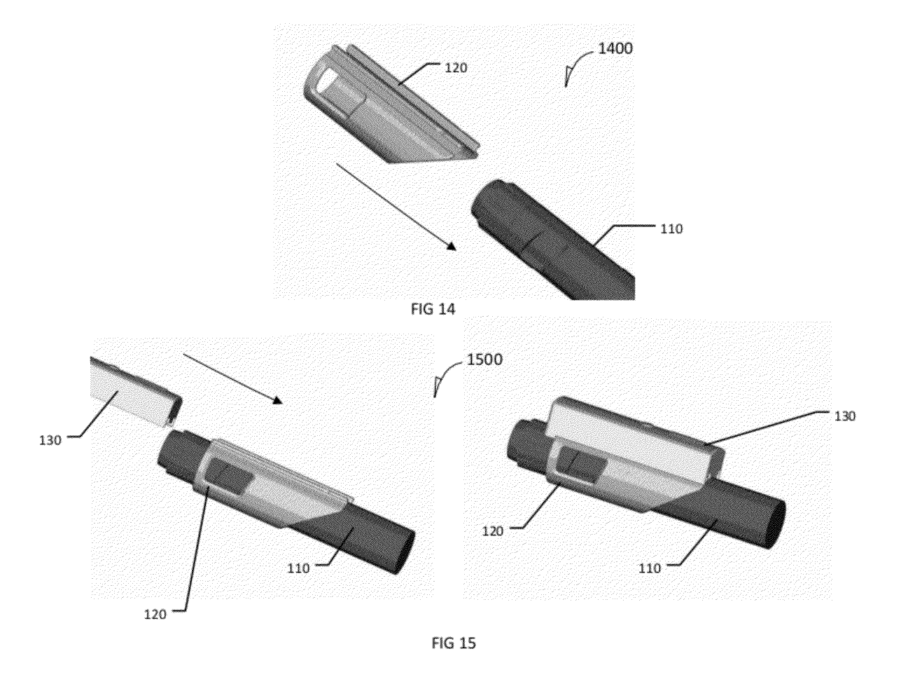

FIG. 12 shows a sleeve being mounted on a Sanofi Aventis Lantus SoloStar medication pen, in accordance with an example embodiment. FIG. 13 shows a universal header being installed on a sleeve mounted on a Sanofi Aventis Lantus SoloStar medication pen, in accordance with an example embodiment. FIG. 14 shows a sleeve being mounted on a Novo Nordisk Flexpen medication pen, in accordance with an example embodiment. FIG. 15 shows a universal header being installed on a sleeve mounted on a Novo Nordisk FlexPen medication pen, in accordance with an example embodiment.

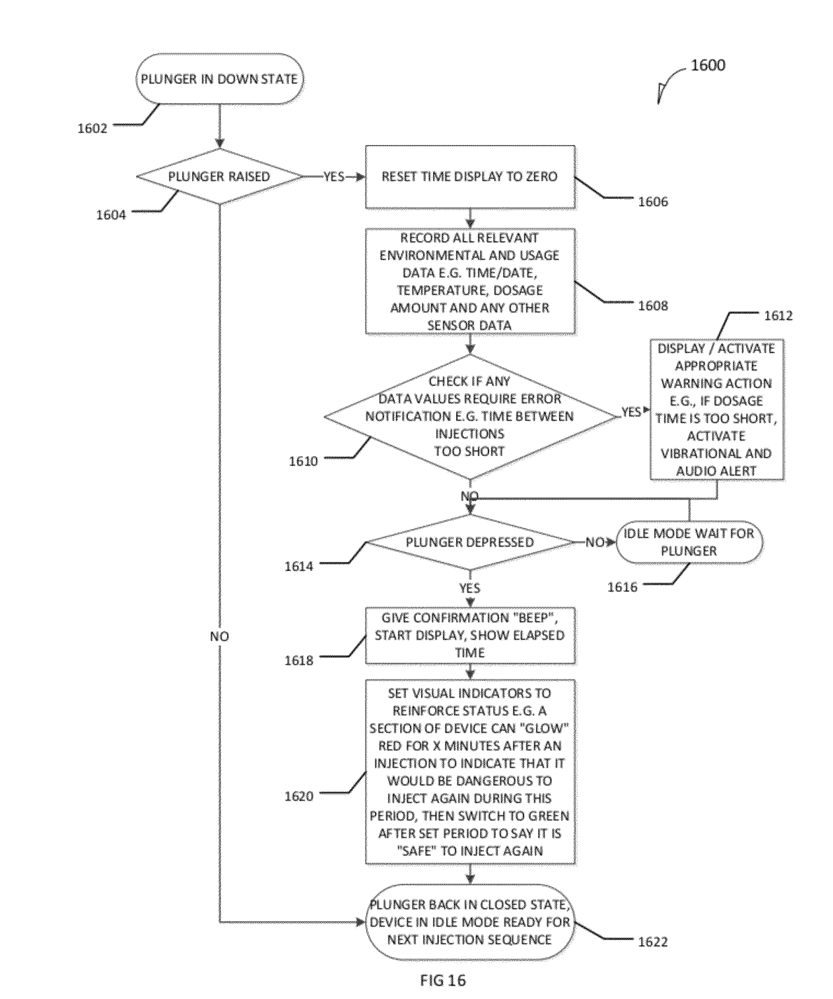

FIG. 16 is a flow diagram showing a method 1600 for supervising injections using the medication injection supervisor device 100, in accordance with an example embodiment. The method can commence at operation 1602 when the plunger 112 is in the down state. At decision block 1604, it can be determined whether or not the plunger 112 is raised. If the plunger 112 is raised at operation 1602 to dial a dosage, the method 1600 can proceed to operation 1606, where the time display is reset to zero, thus starting the time count from the last injection. At operation 1608, relevant environmental and usage data can be recorded. The relevant environmental and usage data can include time, date, temperature, dosage amount, and other sensor data.

At decision block 1610, it is determined, based on the comparison of the recorded environmental and usage data to predetermined optimal values, whether or not an error notification needs to be issued. For example, if the time between injections is too short, an error can be communicated to the user. Thus, if at decision block 1610 it is determined that an error notification needs to be made, the method 1600 proceeds to operation 1612 where the error is displayed via the display 132. In some example embodiments, other appropriate warning actions can be taken. For example, if the time interval between two consecutive injections is too short, vibrational and/or audio alerts can be activated.

If, on the other hand, it is determined at decision block 1610 that no error notification needs to be made, the error is not communicated. In either case, the method 1600 can proceed and at decision block 1614 it can be determined whether the plunger 112 is depressed. If the plunger 112 is not depressed, the method 1600 remains in the idle mode at operation 1616 waiting for the plunger 112 to be depressed. Once the plunger 112 is depressed, the method 1600 can proceed to operation 1618, where the display 132 can start measuring time from the last injection by showing the elapsed time. Additionally, a confirmation “beep” can be provided.

At operation 1620, visual indicators can be set to reinforce the status. For example, a section of the device can “glow” red for a predetermined time interval after the injection to indicate that it would be dangerous to inject again during this period. Once the predetermined time interval has elapsed, the “glow” can become green indicating that it is safe to inject again. Additionally, if the elapsed time is further exceeded past a point where another injection is expected but has not occurred, the “glow” can change to yellow.

FIG. 17 shows a readable plunger subsystem, in accordance with an example embodiment. As shown in FIG. 17, a secondary digitally readable plunger subsystem can be physically attached to the plunger head via a pen specific clip 1710 so that this secondary plunger moves in parallel with the injection pen plunger 112. As the secondary plunger slides in and out of the universal header 130, an electronics assembly inside the header 130 can read linear positions of the secondary plunger and thus infer the dosage of the medication. During the motion of the secondary plunger, the display 132 can show the user the inferred dosage for confirmation. The readings taken by the electronics assembly can be also used to time the occurrences of injections.

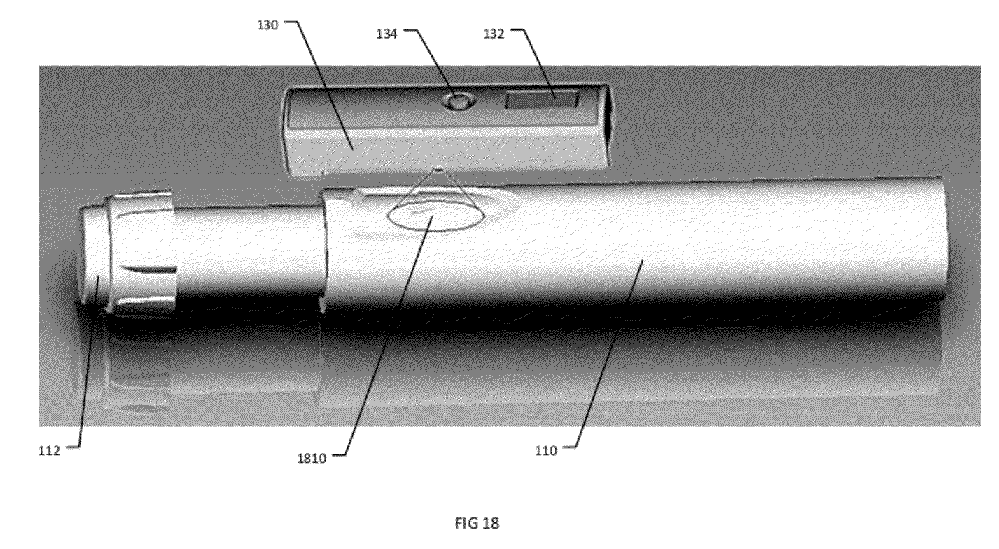

FIG. 18 shows a micro optics and imaging system, in accordance with an example embodiment. As shown in FIG. 18, the micro optics and imaging system can be incorporated in the universal header 130. The sleeve (not shown) can be designed to ensure that the universal header 130 is positioned over the physical dosage dial of the injection pen 110. When the sensor (not shown) is triggered to indicate that an injection is about to start, an imaging system 1810 can be activated and an image recognition performed by reading the maximum dial value, which is then stored in the memory of the medication injection supervisor device 100. During the imaging sequence, the raw data can also be displayed on the display 132 to allow the user to the actual dial value.

As explained above, the medication injection supervisor device or, more specifically, its universal header, is configured for attachment to a prefabricated injection pen prior to use of the pen. The medication injection supervisor device may be attached to the pen during manufacturing of one or both of these components or during subsequent assembly of the two prefabricated components (i.e., the medication injection supervisor device and prefabricated injection pen). For example, a medical professional may attach a medication injection supervisor device to the prefabricated injection pen prior to use of the pen. In certain embodiments, the same type of medication injection supervisor device may be used on different types of prefabricated injection pens (for example, prefabricated injection pens that have different shapes and dimensions). A medication injection supervisor device may be equipped with one or more attachment features for attaching to these types of prefabricated injection pens. In more specific embodiments, the one or more attachment features of the medication injection supervisor device are replaceable such that the attachment features are selected and attached to a device based on the type of the prefabricated injection pen.

Attachment of the medication injection supervisor device to the prefabricated injection pen may be removable or permanent. For purposes of this document, a removable attachment is defined as an attachment of the medication injection supervisor device and prefabricated injection pen that allows for separating these two components later without damaging the components. A permanent attachment is defined as an attachment of the medication injection supervisor device and prefabricated injection pen that does not allow for later separation of these components without damaging one or both components. For example, a universal header of the medication injection supervisor device and the adjacent portion of the prefabricated injection pen may be formed during the same injection molding operation, thereby forming a joined body that is a part of the universal header and prefabricated injection pen. In another example, one component (e.g., a universal header of the medication injection supervisor device) may be molded over a portion of the prefabricated injection pen. No parts of the prefabricated injection pen are added during this overmolding operation. Overmolding forms attachment features in a form of a shell that fully or partially encloses the prefabricated injection pen. In certain embodiments, a universal header of the medication injection supervisor device is glued to the prefabricated injection pen. This embodiment will now be described in more detail with reference to FIG. 19.

FIG. 19 is a cross-sectional schematic view of medication injection supervisor assembly 1900 in which universal header 1902 of the medication injection supervisor device is attached to prefabricated injection pen 1904 using adhesive 1906, in accordance with an example embodiment. Selection of adhesive 1906 depends on materials used for universal header 1902 and prefabricated injection pen 1904. Some examples of adhesives include drying adhesives that harden by solvents (e.g., solvent based adhesives and polymer dispersion adhesives); pressure sensitive adhesives (PSA) that form a bond by the application of pressure; contact adhesives; hot adhesives that are applied in molten form and solidify on cooling (e.g., ethylene-vinyl acetate); multi-component adhesives that harden when their two or more components chemically react (e.g., polyurethanes, epoxies); and one-part adhesives that harden via a chemical reaction by applying radiation, heat, and moisture (e.g., UV cured adhesive).

In certain embodiments, the surface 1903 of universal header 1902 and/or surface 1905 of prefabricated injection pen 1904 that comes in contact with adhesive 1906 is specially treated (e.g., primed) in order to improve the bond between the surface 1903, 1905 and adhesive 1906. An adhesive generally forms a permanent bond between universal header 1902 and prefabricated injection pen 1904. However, in certain embodiments, an adhesive may be used to form a removable attachment. For example, a hot melt adhesive may be reheated to separate universal header 1902 from prefabricated injection pen 1904. In certain embodiments, a double-sided tape may be used instead of or in addition to an adhesive.

A removable attachment may be used for multiple uses of a medication injection supervisor device with different prefabricated injection pens. Generally, a pen is consumed in one or multiple uses during a short period of time, while a medication injection supervisor device may be used for substantially longer periods of time (for example, until the battery dies). In certain embodiments, the power source (e.g., the battery) of the medication injection supervisor device may be recharged or replaced, and the medication injection supervisor device may be used substantially indefinitely. Therefore, instead of providing a medication injection supervisor device with each prefabricated injection pen, a single medication injection supervisor device may be used on multiple pens. A medication injection supervisor device may be designed for use with one type of prefabricated injection pen or multiple types of prefabricated injection pens. When a medication injection supervisor device is switched between different types of prefabricated injection pens, the device may be reconfigured for specific aspects of new prefabricated injection pens. Some examples of removable attachments include sleeves for receiving prefabricated injection pens, slot-ridge attachment mechanisms, pin-cavity attachment mechanisms, cavities in universal headers for receiving clips of the prefabricated injection pens, rings for receiving and compressing around prefabricated injection pens, and various other attachment features. These examples will now be described in more details with reference to corresponding figures.

FIG. 20 is a cross-sectional schematic view of a medication injection supervisor assembly 2000 in which universal header 2002 is attached to prefabricated injection pen 2004 using a slot-ridge attachment mechanism, in accordance with a different example embodiment. Specifically, side edges 2003 of universal header 2002 form a slot for receiving ridge 2006 of prefabricated injection pen 2004. In other embodiments (not shown), a slot may be provided on the prefabricated injection pen while a ridge may be provided on the universal header. For brevity, the following description is focused on a slot provided on the universal header and a ridge provided on the prefabricated form, as shown in FIG. 20. However, one having ordinary skills in the art would understand that the features described below would apply to both configurations.

In certain embodiments, the slot formed by side edges 2003 has an opening that is narrower than bottom surface 2005 of the slot as shown in FIG. 20. Ridge 2006 of prefabricated injection pen 2004 may follow this cross-sectional profile of the slot and have a tapered shape with a neck 2007 narrower than top surface 2008 facing universal header 2002 or, more specifically, bottom surface 2005 of universal header 2002. This configuration of the slot and ridge prevents the ridge from sliding out of the slot in the direction substantially parallel to bottom 2005 of the slot (i.e., the Z direction, as shown in FIG. 20).

The slot-ridge attachment mechanisms may also have features for preventing the ridge from moving with respect to the slot in the direction substantially parallel to the bottom of the slot. For example, bottom surface 2005 of the slot of universal header 2002 and/or top surface 2008 of prefabricated injection pen 2004 may be high friction surfaces, which are sometimes referred to as friction-generating surfaces, when a sheer force is applied along these surfaces. For example, one or both of these surfaces may be made from rubber or rubber-like materials or may have a surface roughness or surface features for preventing one surface from sliding with respect to another surface. In the same or other embodiments, universal header 2002 and/or prefabricated injection pen 2004 include other types of retaining features that prevent these two components from sliding in the Z direction with respect to each other. For example, side edges 2003 of universal header 2002 may have protrusions for engaging with corresponding indents on ridge 2006 of prefabricated injection pen 2004. In certain embodiments, side edges 2003 of universal header 2002 are flexible in order to allow these retaining features to disengage and allow universal header 2002 to move with respect to prefabricated injection pen 2004 in the Z direction (for example, to completely disengage universal header 2002 from prefabricated injection pen 2004). In certain embodiments, these retaining features may allow for adjusting and, for example, locking a position of universal header 2002 with respect to prefabricated injection pen 2004 in the Z direction. This adjustment may be used when different types of prefabricated injection pens are used with the same universal header.

In the same or other embodiments, flexibility of side edges 2003 of universal header 2002 may be used to remove ridge 2006 out of the slot formed by side edges 2003 of universal header 2002 in the Y direction without sliding ridge 2006 out of the slot in the Z direction. Specifically, side edges 2003 of universal header 2002 may flex to widen the opening such that top surface 2008 of ridge 2006 may fit through the opening.

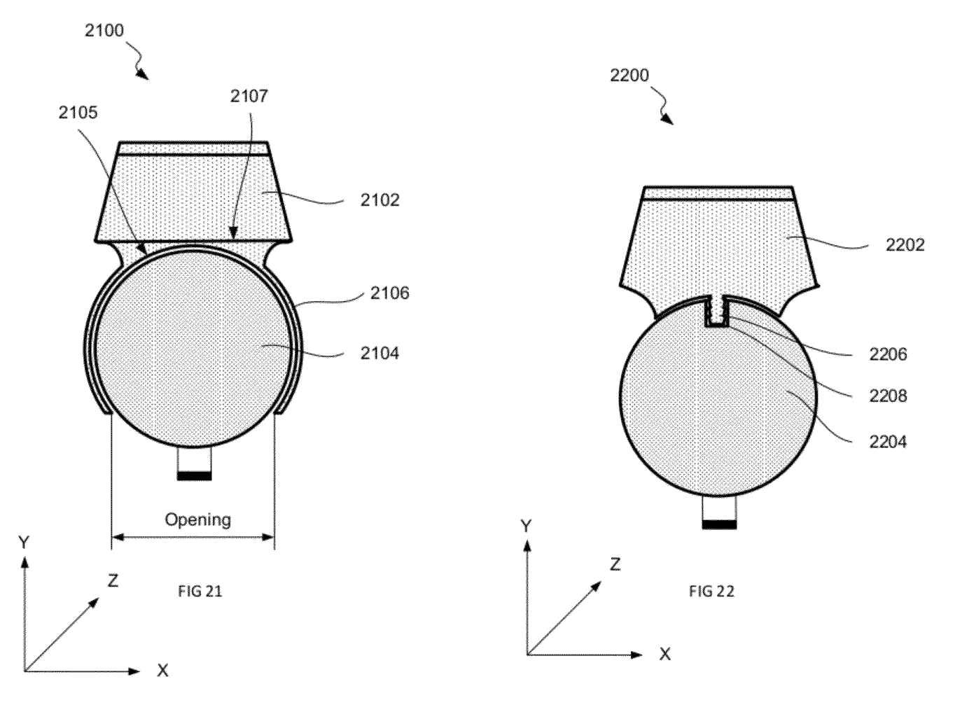

FIG. 21 is a cross-sectional schematic view of a medication injection supervisor assembly 2100 in which universal header 2102 includes sleeve 2106 for supporting universal header 2102 on prefabricated injection pen 2104, in accordance with a different example embodiment. In certain embodiments, sleeve 2106 has an inner surface 2105 that is configured to come in contact with prefabricated injection pen 2104 when universal header 2102 is attached to prefabricated injection pen 2104. Inner surface 2105 may be a friction-generating surface. The friction-generating surface may help to prevent universal header 2102 from sliding with respect to prefabricated injection pen 2104 along the center axis of sleeve 2106 (i.e., in the Z axis direction). In the same or other embodiments, the friction generating surface may help to prevent universal header 2102 from rotating with respect to prefabricated injection pen 2104, when sleeve 2106 and prefabricated injection pen 2104 have substantially round shapes.

Inner surface 2105 of sleeve 2106 may have a profile corresponding to the outer surface of prefabricated injection pen 2104, as shown in FIG. 21. In other words, inner surface 2105 follows the shape of prefabricated injection pen 2104. This configuration increases the contact surface between sleeve 2106 and prefabricated injection pen 2104 and may help to maintain a stronger mechanical attachment between the two.

In certain embodiments, sleeve 2106 is removably attached to universal header 2102. For example, sleeve 2106 may separate from universal header 2102 along interface 2107, as shown in FIG. 21. Various header attachment features many be provided to form this removable attachment. These header attachment features are generally different components from attachment features that are used for attaching the whole medication injection supervisor device to the prefabricated injection pen. Similar to sleeves, other attachment features, such as the slot-ridge mechanisms described above, may be removably attached to the universal header. In certain embodiments, the attachment mechanism is provided on the prefabricated injection pen instead of the medication injection supervisor device. For example, a sleeve may be provided on the prefabricated injection pen, and the medication injection supervisor device may be slid or otherwise inserted into the sleeve.

Returning to FIG. 21, sleeve 2106 may include two flexible portions forming a partial circumference having an opening. These flexible portions may flex in one or more directions (e.g., the X direction) to change the size of the opening. This flexibility may be used for inserting prefabricated injection pen 2104 into sleeve 2106 by pushing prefabricated injection pen 2104 through the opening instead of sliding prefabricated injection pen 2104 into sleeve 2106 along the axis of sleeve 2106. Flexibility of these two flexible portions may also be used to exert some force onto sliding prefabricated injection pen 2104 when it is attached to the medication injection supervisor device in order to provide a more robust mechanical attachment.

In certain embodiments (not shown), a sleeve forms a complete closed loop and a prefabricated injection pen is inserted into such a sleeve by sliding the prefabricated injection pen along the axis of the sleeve. The closed loop sleeve may be mechanically stronger than the open loop sleeve and provide a stronger mechanical attachment, in certain embodiments. When sleeves are used for the attachment of the medication injection supervisor device to the prefabricated injection pen, the sleeve generally extends in the Z direction along the length of the pen. One particular example of a sleeve that only nominally extends in that direction is a ring. In certain embodiments, one or more attachment features comprise at least two rings affixed to the universal header and configured for receiving and tightly fitting the prefabricated injection pen into the two rings. These rings may be spring rings that are configured to change their diameters upon inserting forces onto the two rings when inserting the prefabricated injection pen into the two rings.

FIG. 22 is a cross-sectional schematic view of a medication injection supervisor assembly 2200 in which universal header 2202 of the medication injection supervisor device is attached to prefabricated injection pen 2204 using a cavity-pin coupling, in accordance with a different example embodiment. Specifically, FIG. 22 illustrates universal header 2202 having pin 2206 protruding into cavity 2208 formed in prefabricated injection pen 2204. Universal header 2202 and prefabricated injection pen 2204 may have one or more such cavity-pin couplings. For example, two or more cavity-pin couples may be provided along the length of prefabricated injection pen 2204 (i.e., the Z direction). It should be noted that pins may be provided on the universal header of the medication injection supervisor device, as shown in FIG. 22, or on the prefabricated injection pen (not shown). In the latter embodiments, the corresponding cavities are provided in the universal header. A cavity-pin coupling may include various retaining features to prevent the pin from sliding out of the cavity.

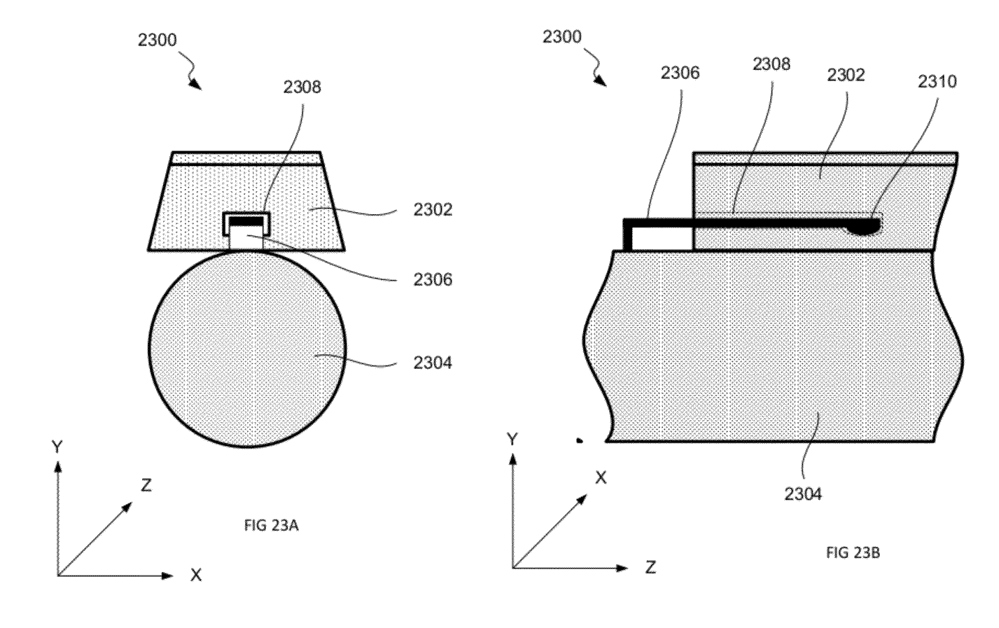

FIG. 23A is a cross-sectional schematic view of a medication injection supervisor assembly 2300 in which universal header 2302 of the medication injection supervisor device is supported on prefabricated injection device 2304 by sliding clip 2306 of prefabricated injection device 2304 into cavity 2308 of universal header 2302, in accordance with a different example embodiment. FIG. 23B is a side schematic view of the same medication injection supervisor assembly 2300 shown in FIG. 23A. Clip 2306 of prefabricated injection device 2304 may be similar to a clip of a writing pen and be used, when not attached to the medication injection supervisor device, for supporting the prefabricated injection pen in a pocket and the like. Clip 2306 may be flexible and exert some force when pulled away from prefabricated injection device 2304. This force may be used to ensure a stronger mechanical attachment of the medication injection supervisor device on prefabricated injection device 2304. In certain embodiments, a free end of clip 2306 may include a shaped portion or protrusion 2310 for engaging with a corresponding shaped portion or cavity of universal header 2302. These features may be used to prevent universal header 2302 from sliding from clip 2306 in the direction along length of clip 2306 (i.e., the Z direction).

Thus, example embodiments of a medication injection supervisor device have been described. Although embodiments have been described with reference to specific example embodiments, it will be evident that various modifications and changes may be made to these embodiments without departing from the broader spirit and scope of the system and method described herein. Accordingly, the specification and drawings are to be regarded in an illustrative rather than a restrictive sense.

What is claimed is:

1. A medication injection supervisor device comprising:
a universal header comprising one or more attachment features for attaching the universal header to a prefabricated injection pen;
an electronics assembly housed by the universal header;
a sensor to automatically detect an injection, the sensor being communicatively coupled to the electronics assembly;
a display to display injection data; and
one or more buttons to allow manipulation and display of the injection data, the manipulation including resetting a time.

2. The medication injection supervisor device of claim 1, wherein the one or more attachment features are configured to provide removable attachment of the universal header to the prefabricated injection pen.

3. The medication injection supervisor device of claim 2, wherein the one or more attachment features comprise a sleeve having an inner surface; and
wherein the inner surface is configured to come in contact with the prefabricated injection pen when the universal header is attached to the prefabricated injection pen.

4. The medication injection supervisor device of claim 3, wherein the inner surface comprises a friction-generating surface.

5. The medication injection supervisor device of claim 3, wherein the inner surface follows a shape of the prefabricated injection pen.

6. The medication injection supervisor device of claim 3, wherein the sleeve is removably attached to the universal header.

7. The medication injection supervisor device of claim 3, wherein the sleeve comprises two flexible portions forming a partial circumference having an opening; and
wherein flexing the flexible portions changes a size of the opening.

8. The medication injection supervisor device of claim 2, wherein the one or more attachment features comprise a slot for receiving a ridge of the prefabricated injection pen.

9. The medication injection supervisor device of claim 8, wherein an opening of the slot is narrower than a bottom of the slot to prevent the ridge from sliding out of the slot in a direction substantially perpendicular to the bottom.

10. The medication injection supervisor device of claim 8, wherein the slot comprises one or more retaining features to prevent the ridge from sliding out of the slot in a direction substantially parallel to the bottom of the slot.

11. The medication injection supervisor device of claim 2, wherein the universal header comprises a cavity for receiving a clip of the prefabricated injection pen.

12. The medication injection supervisor device of claim 2, wherein the universal header comprises a cavity for receiving a pin of the prefabricated injection pen.

13. The medication injection supervisor device of claim 2, wherein the universal header comprises a pin for inserting into a cavity of the prefabricated injection pen.

14. The medication injection supervisor device of claim 2, wherein the one or more attachment features comprise at least two rings affixed to the universal header and configured for receiving and tightly fitting the prefabricated injection pen into the two rings.

15. The medication injection supervisor device of claim 14, wherein the two rings are spring rings configured to change their diameters upon inserting forces onto the two rings when inserting the prefabricated injection pen into the two rings.

16. The medication injection supervisor device of claim 1, wherein the universal header comprises a surface for contacting the prefabricated injection pen; and
wherein the surface conforms to a profile of the prefabricated injection pen.

17. The medication injection supervisor device of claim 16, wherein the one or more attachment features comprise a double-sided tape positioned on the surface of the universal header.

18. A medication injection supervisor assembly comprising:
a prefabricated injection pen;
a universal header permanently attached to the prefabricated injection pen;
an electronics assembly housed by the universal header;
a sensor to automatically detect an injection, the sensor being communicatively coupled to the electronics assembly;
a display to display injection data; and
one or more buttons to allow manipulation and display of the injection data, the manipulation including resetting a time.

19. The medication injection supervisor assembly of claim 18, wherein the universal header is permanently attached to the prefabricated injection pen using an adhesive.

20. The medication injection supervisor assembly of claim 18, wherein the universal header forms a joined body with the prefabricated injection pen.
